(19) United States
(12) Patent Application Publication
(10) Pub. No.: US 2023/0277042 A1
(43) Pub. Date: Sep. 7, 2023

(54) DEVICE FOR COUPLING COHERENT LIGHT INTO AN ENDOSCOPIC SYSTEM

(71) Applicant: LIMIS DEVELOPMENT B.V., Leeuwarden (NL)

(72) Inventors: Joost Emiel Marcel Calon, Gorredijk (NL); Joris Sebastiaan De Graaf, 's-Hertogenbosch (NL)

(21) Appl. No.: 18/019,713

(22) PCT Filed: Aug. 6, 2021
(86) PCT No.: PCT/EP2021/072041

§ 371 Date: Feb. 3, 2023

(30) Foreign Application Priority Data
Aug. 7, 2020 (NL) 2026240

(51) Int. Cl. A61B 1/00; A61B 1/07; A61B 1/06; A61B 1/05

(52) U.S. Cl. CPC A61B 1/00126; A61B 1/07; A61B 1/00117; A61B 1/0655; A61B 1/063; A61B 1/05; A61B 1/00009; A61B 1/0661

(57) ABSTRACT

An endoscopic system comprises an incoherent light source. A coupling device comprises a first light source generating coherent light of a first wavelength, or a coherent light input receiving light of the first wavelength. The coupling device comprises an input with a connector for releasably connecting to the incoherent light source via a second light guide, and an output comprising a connector for releasably connecting to an endoscope via the first light guide. The input receives light from the endoscopic system. The output provides incoherent light and coherent light to the first light guide. The coupling device may be configurable in a first state being an unobstructed light beam path for propagating the incoherent light from the input to the output; and in a second state being an unobstructed light beam path for propagating the coherent light from the coherent light source or the coherent light input to the output.

### CROSS-REFERENCE TO RELATED APPLICATIONS

This application is a Section 371 National Stage Application of International Application No. PCT/EP2021/072041, filed Aug. 6, 2021 and published as WO 2022/029308 A1 on Feb. 10, 2022, in English, and further claims priority to Netherlands Application No. 2026240, filed Aug. 7, 2020 the contents of which are hereby incorporated by reference in their entirety.

### FIELD OF THE INVENTION

The disclosure relates to coupling coherent light into a light delivery system of an endoscopic system and in particular, though not exclusively, to devices, systems and methods for coupling coherent light into a light delivery system of an endoscopic system and to computer program products using such methods.

### BACKGROUND

Endoscopic systems are used to view or image regions within a human or animal body, typically during medical procedures, e.g., surgery or diagnostics. For the scope of this disclosure, endoscopic systems may include systems for internal inspection of both naturally accessible body parts and cavities with artificially created openings, e.g., laparoscopic systems, arthroscopic systems, bronchoscopic systems, neuroendoscopic systems, et cetera. Endoscopic systems typically include an endoscope comprising a light delivery system and an image guide, a white light source, and an imaging device such as a camera head. Typically, an endoscope includes a rigid or flexible elongated insertion tube equipped with a first light guide, e.g., a fiber bundle, to illuminate a target, and a second light guide, e.g., a rod lens, to transfer an image of the target to the imaging system. Other endoscopes may have an imaging sensor at or near the tip, so-called chip-on-the-tip systems. The endoscope is connectable to the (external) light source and a screen using cables. The white light illumination provides a surgeon or endoscopist with an easy to interpret image.

Many modem medical imaging techniques require coherent light, e.g., laser light. For example, laser speckle contrast imaging (LSCI), sometimes referred to as laser speckle contrast analysis (LASCA), uses coherent light to provide a fast, full-field, cheap, and relatively simple in vivo imaging method for determining two-dimensional perfusion maps of living biological tissue. Perfusion can be an indicator for tissue viability and thus may provide valuable information during diagnostics and surgery. As a further example, fluorescent imaging, e.g., indocyanine green (ICG) based perfusion imaging, may require coherent light to excite fluorescent markers.

It is therefore desirable to combine the advantages of white light imaging and coherent light imaging using a single endoscope.

WO2015/176294A1 describes a laparoscopic system for imaging subsurface blood flow of tissue based on laser speckle contrast imaging. The system comprises a white light source, a laparoscope with a light guide for transmitting the white light to a field of view, a laser light source, and an optical fiber to transmit the laser light to the field of view. The optical fiber may be inserted in a body of a patient via a separate incision, or the optical fiber may be integrated as a separate channel into or onto a shaft of the laparoscope, which requires replacing or at least substantially altering the laparoscope.

US2016/0022126A1 describes an endoscopic system with a single light source which is capable of providing white light and infrared laser light for exciting fluorescent markers. However, using this system would result in replacing the entire endoscopic system, or at the very least the entire light source. This is relatively expensive.

Hence, from the above, it follows that there is a need in the art for a system to combine the advantages of coherent light imaging with an existing endoscopic system.

### SUMMARY

It is an objective of the embodiments in this disclosure to reduce or eliminate at least one of the drawbacks known in the prior art.

In a first aspect, the invention relates to a coupling device for coupling coherent light and incoherent light into a first light guide of a light delivery system of an endoscopic system, the endoscopic system comprising an incoherent light source for generating the incoherent light, e.g., white light. The coupling device may comprise an optical input comprising an optical input connector for connecting, preferably releasably connecting, the coupling device to the incoherent light source via a second light guide, the optical input being configured for receiving the incoherent light from the endoscopic system; a first light source for generating coherent light of a first wavelength, or a coherent light input for receiving coherent light of the first wavelength; and an optical output comprising an optical output connector for releasably connecting the coupling device to an endoscope via the first light guide, the optical output being configured for providing the incoherent light and the coherent light of the first wavelength to the first light guide. In an embodiment, the coupling device may be configurable in a first state and a second state, in which first state, the coupling device may comprise an unobstructed incoherent light beam path for propagating the incoherent light from the optical input to the optical output; and in which second state, the coupling device may comprise an unobstructed coherent light beam path for propagating the coherent light from the coherent light source or from the coherent light input to the optical output. The coupling device may further comprise an optical coupler for, simultaneously and/or alternately, injecting the incoherent light and the coherent light of the first wavelength into the first light guide, wherein the coherent light and the incoherent light have a shared optical path at the optical output.

The optical output comprises a single output channel, possibly comprising a plurality of fibers, for providing the incoherent light and/or the coherent light to the first light guide of the light delivery system of the endoscopic system. Thus, the coherent light and the incoherent light have a shared optical path at the optical output of the coupling device and consequently at the entrance of the first light guide. The first state and the second state may fully or partially overlap, resulting in a state in which both the incoherent light and the coherent light share the optical path in a simultaneous manner. The first state and the second state may also be distinct states, resulting in a time-division manner of sharing the optical path.

In some cases, a cable comprising the first light guide may comprise additional channels. The coupling device may comprise additional outputs (i.e., in addition to the optical output) for providing input to such additional channels.

As used herein, light being provided to a light guide means that the light is injected into a light entrance of the light guide. When light is provided to an optical output, it is understood that the light is also provided to a light guide connected to the optical output, when present.

The incoherent light may be white light or colored light, e.g., blue light. The incoherent light source may also comprise a plurality of coherent light sources providing coherent light simultaneously or alternately, e.g., in a time-division manner. The endoscopic system may be any system for internal visual inspection of objects, preferably human or animal bodies, preferably living bodies. Internal visual inspection may relate both to naturally accessible body parts such as the bladder, intestines, or bronchia, and to cavities with artificially created openings such as the abdominal cavity or with neurosurgery. Thus, the endoscopic system may be, e.g., a laparoscopic system, an arthroscopic system, a bronchoscopic system, a neuroendoscopic system, et cetera. The endoscopic system may further comprise surgical instruments, e.g., a robotic surgery system.

By connecting the coupling device between the incoherent light source of the endoscopic system and an endoscope proper, coherent light may be guided into first light guide of the light delivery system of the endoscope, thus allowing for additional imaging capabilities, such as LSCI and/or fluorescence imaging, e.g., indocyanine green (ICG) imaging for perfusion imaging or angiography. Other fluorescent markers may be used for other applications. For example, fluorescence imaging may also be used to detect tumor tissue. In general, different markers are activated by light of different wavelengths.

By using a separate device, there is no need to replace the light source or even the entire endoscopic system, or to disconnect and reconnect the first light guide of the light delivery system each time a user wishes to switch between an incoherent light source and a coherent light source. Thus, it is a relatively economical, flexible, and easy to operate option.

The coupling device is connectable to the first light guide of light delivery system, rather than directly to a light input of the endoscope proper. This way, the coupling device can be placed outside a sterile zone in e.g., an operation theatre. This way, the device does not need to be sterilized before each use, increasing ease of use and reducing both production costs and operating costs. By contrast, a device that would be attached directly to an endoscope would need to be compatible with conventional sterilization methods. This would be particularly problematic for embodiments comprising a coherent light source, which may be sensitive to, e.g., heat and disinfectants.

However, because the coupling device is connectable to the endoscope via the first light guide, the light needs to be coupled into the first light guide with sufficient intensity. This is particularly relevant for the incoherent light. Therefore, the coupling device may be configured to minimize light losses, in particular incoherent light losses, between the optical input and the optical output.

The coherent light may be generated in the coupling device itself, or may be generated by a different device and provided to the coupling device using e.g., an optical fiber. Using a coherent light input may result in a smaller device which may be placed in a mostly unrestricted way in an endoscopy system. Additionally, different coherent light sources may be attached based on different needs, e.g., for different fluorescent markers.

On the other hand, using a first coherent light source inside the coupling device may result in higher quality coherent light (e.g., higher power, smaller spectral bandwidth and/or a longer coherence length), as the coherent light may propagate through free space to the optical output or at least to the optical coupler, without having to use optical components such as optical fibers which may be detrimental to the quality of the coherent light. As is described below in more detail, the optical coupler may additionally comprise a second coherent light source and/or a coherent light input for receiving coherent light of a second wavelength.

The optical coupler may be a static optical coupler for combining a relatively wide incoherent light bundle and a relatively narrow coherent light bundle on a single optical path, and injecting both the incoherent light and the coherent light into a first light guide of a light delivery system of an endoscopic system via an optical output. Alternatively, the optical coupler may be an optical switch for (selectively) injecting either the coherent light bundle or the incoherent light bundle or both into the first light guide.

In an embodiment, the optical coupler may be an optical switch configured for switching between a first state wherein the coherent light is not injected into the first light guide, and a second state wherein the coherent light is injected into the first light guide. This way, an operator may select whether or not to use the laser light, without having to remove the coupling device.

In an embodiment, in the first state, the optical switch may be configured to provide the incoherent light at the optical output. In the second state, the optical switch may be configured to not provide the incoherent light at the optical output. In many applications, a user may want to switch between only incoherent light and only coherent light.

In an embodiment, the optical switch may be configured for switching to a third state, in which third state the optical switch may be configured to provide both the coherent light and the incoherent light at the optical output. Imaging a target area with both coherent and incoherent light simultaneously may be particularly advantageous when the coherent light is outside the visible spectrum, e.g., infrared or ultraviolet light.

In an embodiment, the optical switch may comprise a first movable, e.g., rotatable, mirror, the mirror being movable between a first position in which a reflective surface of the first mirror is positioned to reflect incoherent light propagating over the incoherent light beam path, and a second position, in which the reflective surface of the first mirror is positioned to reflect the incoherent light in a direction away from the incoherent light beam path. Additionally or alternatively, the optical switch may comprise a second mirror movable between a first position in which a reflective surface of the second mirror is positioned to reflect coherent light propagating over the coherent light beam path, and a second position, in which the reflective surface of the first mirror is positioned to reflect the coherent light in a direction away from the coherent light beam path.

The second mirror may be the same mirror as the first mirror, a different part of the same mirror, or a separate mirror. Moving mirrors may be cheap and easy to manufacture.

In an embodiment, the optical switch may comprise a first switchable mirror, switchable between a transparent state and a reflective state, positioned to reflect, in the reflective state, incoherent light propagating over the incoherent light beam path. Additionally or alternatively, the optical switch may comprise a second switchable mirror, switchable between a transparent state and a reflective state, positioned to reflect, in the reflective state, coherent light propagating over the coherent light beam path.

The first and/or second switchable mirror may be, e.g., an electrically switchable mirror. Switching a mirror between a transparent state and a reflective state may be faster than moving the mirror, and the absence of moving parts may reduce pollution of optical elements in the optical switch. The first and second switchable mirrors may be the same mirror or may be different mirrors.

In an embodiment, the optical switch may comprise a first light beam blocker configured to block the coherent light in the first state, preferably the first beam blocker comprising a mechanical shutter, preferably a rotating shutter, more preferably a motorized rotating shutter, or an optical beam blocker, preferably one of: a liquid crystal light valve, an acousto-optical modulator, an electro-optical modulator or a photo-elastic modulator.

Optionally, the optical switch may further comprise a second light beam blocker configured to block the incoherent light in the second state, preferably the second beam blocker comprising a mechanical shutter, preferably a rotating shutter, more preferably a motorized rotating shutter, or an optical beam blocker, preferably a liquid crystal light valve.

Mechanical shutters are easy and cheap to manufacture and operate. In some embodiments, both the coherent light and the incoherent light may be switched using shutters. By using rotating shutters in counter phase, alternating illumination with incoherent light and coherent light may be obtained at a relatively high frame rate. This may allow for e.g., multiplexing coherent light images and incoherent light images.

Optical beam blockers do not comprise moving parts and are thus dust-free.

They are also typically fast and relatively simple to operate electronically.

In an embodiment, the optical coupler may comprise a first convergent lens to reduce divergence of the incoherent light from the optical input and a second convergent lens to focus the incoherent light into the first light guide (or, on the optical output); and a mirror, positioned between the first lens and the second lens, configured to steer the laser beam into the first light guide (or, on the optical output).

Using lenses to focus the incoherent light into the first light guide results in very little light loss. In an embodiment, one or more lenses may be replaced by mirrors, e.g., a converging lens may be replaced by a concave mirror, optionally comprising a hole or window to allow for an unobstructed coherent light beam path through the concave mirror.

In an embodiment, the optical coupler may comprise a tapered light pipe having a wide end for receiving the incoherent light from the optical input and a narrow end for providing the incoherent light to the optical output. The tapered light pipe may be divided into a first part and a second part by a plane intersecting the tapered light pipe between the wide end and the narrow end, a normal of the plane defining a non-zero angle with a longitudinal axis of the tapered light pipe. Preferably, the tapered light pipe may further comprise a protrusion having a surface normal to the coherent light path for receiving the coherent light. The optical coupler may further comprise a mirror, positioned on the plane between the first part and the second part of the tapered light pipe on the longitudinal axis of the tapered light pipe, configured to steer the laser beam into the first light guide (or, onto the optical output).

Using a tapered light pipe results in little chromatic aberrations. The protrusion may increase the amount of coherent light entering the light guide.

Both the embodiment with a tapered light pipe and the embodiment with lenses allow switching the coherent light and incoherent light independently using mechanical or optical shutters in the respective beam paths. Thus, incoherent light and coherent light may be used either separately or combined.

In an embodiment, the optical switch may comprise an internal light guide for guiding the incoherent light from a first end of the internal light guide to a second end of the internal light guide. The optical switch may further comprise a switching body comprising at least the second end of the internal light guide, the switching body being movable between a first position corresponding to the first state and a second position corresponding to the second state. In the first position, the switching body may be configured to block the coherent light beam path, to position the first end of internal the light guide for receiving incoherent light from the optical input, and to position the second end for injecting the incoherent light into the first light guide. In the second position, the switching body may be configured not to block the coherent light beam path, and to position at least the second end of the internal light guide such that the incoherent light is not injected into the first light guide.

For example, the beam path from the first coherent light source to the optical output may be a path through free space. In the first position, the light guide may be configured to guide the incoherent light to optical output, simultaneously blocking the beam path of the coherent light. By moving the light guide to a different position, the coherent light beam path to the optical output may be unobstructed. This way, very few optical components are used to provide the coherent light at the optical output, and hence, into the first light guide, which is beneficial for the spectrum and coherence length of the coherent light.

The use of an internal light guide for transporting the incoherent light minimizes light loss and chromatic aberrations. Although some incoherent light sources may automatically compensate a reduced incoherent light intensity, this compensation is typically limited. Thus, even is such systems, it is preferably to minimize light loss.

In an embodiment, the internal light guide may be a flexible light guide, preferably a fused fiber bundle or a liquid light guide, and the first end of the internal light guide may be connected to the optical input. By connecting the light guide to the optical input, the number of interfaces between light guide segments may be reduced, which may be beneficial for the light intensity and light quality of the incoherent light.

In an embodiment, the optical switch may be an electrically operated switch with a motor, optionally a servomotor; and the optical switch may be moved between a first position corresponding to the first state and a second position corresponding to the second state. The switching body may be moved via a cam or a crank. In a different embodiment, the optical switch may be operated manually.

A manually operated switch may be simpler and cheaper to manufacture than an electrically operated one. An electrically operated switch may be substantially faster, and easier to remotely operate, than a manually operated one. An electrically operated switch may comprise a motor to move a movable part of the optical switch, e.g., a movable mirror or the switching body, between the first position and the second position. A servomotor may be used to quickly and accurately move the optical switch.

In an embodiment, the optical switch may comprise a first end stop configured to stop the switch in a first position corresponding to the first state; and a second end stop configured to stop the switch in a second position corresponding to the second state. The optical switch may further comprise a spring configured to keep the switch in the first or second position if the switch is not being operated. This configuration is reliable and accurate, and may be cheaper than using a servomotor.

In an embodiment, the optical switch is configured to switch automatically back and forth between the first state and the second state, preferably with a frequency of at least 10 Hz, more preferably with a frequency of at least 30 Hz, even more preferably with a frequency of at least 100 Hz.

By alternating between two illumination states, alternating images may be acquired. This may be beneficial for further processing, where e.g., images based on coherent light imaging may be overlaid on the preceding or subsequent incoherent light image, thus combining information from two imaging modalities.

In an embodiment, the optical input connector is configured for connecting, preferably releasably connecting, to an incoherent light output of the endoscopic system via a light pipe; that is, the second light guide may be a light pipe. In a different embodiment, the optical input connector is configured for connecting, preferably releasably connecting, to an incoherent light output of the endoscopic system via a flexible light guide, preferably a fiber bundle, a fused fiber bundle, or a liquid light guide; that is, the second light guide can be a flexible light guide. A rigid connection using a light pipe results in very little light loss, while a flexible connection using an optical fiber allows for a more flexible placement of the coupling device relative to the incoherent light source of the endoscopic system.

In an embodiment, the first light source is a narrow-bandwidth laser, preferably having a spectrum bandwidth of less than 1 nm, more preferably less than 0.2 nm, even more preferably less than 0.1 nm.

In an embodiment, the first light source may have a coherence length of at least 0.35 mm, preferably at least 1.5 mm, more preferably at least 3.5 mm.

In an embodiment, the first light source may be a laser with a power output of at least 20 mW, preferably at least 100 mW, even more preferably more at least 150 mW.

High-quality laser speckle contrast images may be obtained using a coherent light source with a small spectral bandwidth and long coherence length. A coherent light source with a power output of at least 20 mW may prevent underexposure during imaging, depending on the site to be imaged.

In an embodiment, the first wavelength may be selected in the red part of the electromagnetic spectrum, preferably in the range 600-700 nm, more preferably in the range 630-660 nm. In another embodiment, the first wavelength may be selected in the infrared part of the electromagnetic spectrum, preferably in the range 700-1200 nm, more preferably in the range 700-900 nm, even more preferably in the range 770-790 nm or in the range 820-840 nm.

For laser speckle contrast images, e.g., perfusion images, red light is preferred, as they have a relatively large penetration depth and are well reflected by red blood cells. If an RGB camera or equivalent component of the endoscopic system is used, the first wavelength may be selected to be imaged by the red channel of the RGB camera. Infrared light may also be used for laser speckle contrast imaging, and has a larger penetration depth than red light. Infrared light may also be used for ICG-based fluorescence imaging. Infrared light imaging may be combined with simultaneous white light imaging. A further advantage of using infrared light is that laser speckle contrast imaging and ICG-based fluorescence imaging may be performed simultaneously.

Some endoscopes may comprise filters blocking infrared light and/or may not be configured for detecting infrared light; thus, using coherent light in the visible spectrum may allow for using the coupling device or imaging system with a wider range of endoscopes.

In an embodiment, the coupling device may further comprise a second light source for generating light of a second wavelength; and an optical combiner for combining the light of the first wavelength with the light of the second wavelength, the optical combiner preferably comprising a dichroic mirror for selectively reflecting the light of the first wavelength or the light of the second wavelength.

A second coherent light source may be advantageous for laser speckle contrast images, as images obtained using the second wavelength may be used to correct images obtained using the first wavelength.

In an embodiment, the second wavelength may be selected in the blue or green part of the electromagnetic spectrum, preferably in the range 380-590 nm, more preferably in the range 470-570 nm, even more preferably in the range 520-560 nm.

For laser speckle imaging, it is advantageous if the second wavelength has a small penetration depth and is to a large extent reflected by tissues that are being imaged. If an RGB camera or equivalent component of the endoscopic system is used, the second wavelength may be selected to be imaged by the green or blue channel of the RGB camera. Preferably, the first and second wavelength are far enough apart on the electromagnetic spectrum to limit crosstalk between the RGB channels.

The endoscopic system may further comprise a camera, preferably an RGB camera, the camera being configured to provide a video signal. In an embodiment, the coupling device may further comprise: a video signal input connector for receiving the video signal; a video signal output connector for providing a video output; and an image processing module for generating coherent light images, preferably fluorescence images and/or laser speckle contrast images based on the video signal when the optical coupler injects coherent light into the first light guide.

In general, instead of an RGB camera, dual or triple monochrome cameras, e.g., CCD cameras, may be used with, for example, different color filters, e.g., red and green filters, or a wavelength-selective beam splitter, e.g., a prism. Alternatively, a single monochrome camera with a mechanically, optically, or electrically switchable color filter or other wavelength-based selection device may be used. In such cases, a red channel of an RGB camera may be read as, e.g., a monochrome camera configured to measure light in the red part of the electromagnetic spectrum and, mutatis mutandis, the same for references to the green and blue channels, as the case may be. This relates both to cameras in endoscopic systems and to cameras in imaging systems according to an embodiment of the invention.

In an embodiment, in the first state, the video signal may be looped to the video output. In the second state, the video signal may be provided to the image processing module. The image processing module may be configured to determine one or more output images, preferably laser speckle contrast images or fluorescence images, based on the video signal. The one or more output images may be provided at the video signal output connector.

Including an image processing module prevents the need to use a separate image processing module to process the coherent light images.

In an embodiment, the coupling device may further comprise a frame grabber for converting a continuous video input signal into discrete frames for processing by the video processing module.

In an embodiment, the coupling device may further comprise a first sensor, preferably an optical sensor, and a controller, connected to the first sensor. The first sensor may be configured to send a first signal to the controller only if the first light guide is connected to the optical output connector; and the controller may be configured to switch off or block the first coherent light source and, optionally, the second coherent light source, if the controller does not receive the first signal.

In an embodiment, the coupling device may further comprise a second sensor, preferably an optical sensor, and a controller, connected to the second sensor. The second sensor may be configured to send a second signal if the optical switch is in the second state; and the controller may be configured to switch off or block the first coherent light source and, optionally, the second coherent light source, if the controller does not receive the second signal.

Such sensors may increase the safety of the coupling device, by preventing coherent light from exiting the coupling device if there is the first light guide is not connected to deliver the coherent light.

In an embodiment, the coupling device may further comprise a control switch for generating a trigger signal or a trigger input for receiving a trigger signal, wherein the optical switch is configured to switch from the first state to the second state in response to receiving a trigger signal.

In an embodiment, the coupling device may further comprise a data storage medium or a data output.

The endoscopic systems described in this disclosure are not limited to medical endoscopic systems, but may also include similar systems comprising an incoherent light source and a light delivery system from other fields, e.g., manufacturing, gunsmithing, or aviation. Endoscopic systems in non-medical fields are sometimes referred to as borescopes or fiberscopes. They may be used for visual inspection of e.g., engines or the interior bore of a firearm.

In a further aspect, the invention may relate to an image processing system for generating coherent light images with an endoscopic system. The endoscopic system may comprise an incoherent light source, a video processing unit, and an endoscope, with the endoscope being connected to a camera. In an embodiment, the image processing system may comprise a coupling device according to one of the embodiments described above, and a video processing device. The video processing device may comprise a video signal input connector for receiving a video signal from the camera. The video processing device may further comprise a first video signal output connector for providing a video output, the first video signal output connector configured to be connected to a video signal input of the video processing unit of the endoscopic system. The video processing device may also comprise an image processing module for generating coherent light images based on the video signal when the optical switch is in the first state.

In an embodiment, the image processing system may further comprise a second video signal output connector configured to be connected to a second display, the image processing system being configured to provide an incoherent light image to the first video output when the optical switch is in the first state and to provide a coherent light image to the second video signal output when the optical switch is in the second state.

In an aspect, the invention may relate to an imaging system for generating an infrared coherent light image with an endoscopic system, the endoscopic system comprising an incoherent light source, an endoscope, and a light delivery system for delivering light to the endoscope. In an embodiment, the imaging system may comprise a coupling device as described above, the first wavelength being selected in the infrared part of the electromagnetic system, preferably in the range 700-1200 nm, more preferably in the range 700-900 nm, even more preferably in the range 770-790 nm or in the range 820-840 nm. The imaging system may further comprise an infrared imaging sensor configured to receive light collected by the endoscope, and an image processing module configured to receive a video signal from the infrared imaging sensor and to determine a coherent light image based on the received video signal.

The infrared imaging sensor may be included in an RGB/IR camera or equivalent device, i.e., a camera configured to capture images in both the visible light and the infrared part of the electromagnetic spectrum using a single sensor array. Alternatively, the imaging sensor may further comprise a beam splitter for splitting the light reflected by the target into an infrared part imaged by the infrared imaging sensor, and a visible light part imaged by an RGB camera included in either the endoscopic system or the imaging system, as described above.

In an aspect, the invention may relate to an endoscopic system, preferably a laparoscopic system, comprising the coupling device as described above.

In an aspect, the invention may relate to a method for generating a coherent-light-based image of a target area in a patient body using an endoscopic system. The endoscopic system may comprise an incoherent light source for generating incoherent light, e.g., white light, and an endoscope, the endoscope comprising an insertion tube for inserting in the patient body, a light delivery system for illuminating the target area, and an image sensor for acquiring an image of the target area. The endoscope may be connected, preferably releasably connected, to a coherent light coupling system via a first light guide, the coherent light coupling system being configured to receive the incoherent light from the incoherent light source via a second light guide, receive or generate coherent light of a first wavelength, and selectively provide the coherent light and/or the incoherent light to the first light guide, wherein the coherent light and the incoherent light preferably have a shared optical path in the first light guide. In an embodiment, the method may comprise receiving a first trigger signal and, in response to receiving the trigger signal, providing coherent light to the light delivery system. The method may further comprise receiving a video stream from the image sensor, the video stream comprising a signal representing a light intensity of the coherent light reflected or dispersed by the target area. The method may further comprise determining a coherent light image, e.g., a laser speckle contrast image or fluorescence image, based on the video stream.

In an embodiment, the method may further comprise displaying and/or storing the determined coherent light image.

In an embodiment, the method may further comprise, in response to receiving a second trigger signal, or a predetermined amount of time after receiving the first trigger signal, providing the incoherent light to the light delivery system and, preferably, blocking the coherent light from entering the light delivery system.

The invention may also relate to a computer program or suite of computer programs comprising at least one software code portion or a computer program product storing at least one software code portion, the software code portion, when run on a computer system, being configured for executing one or more of the method steps described above.

The invention may also relate to a non-transitory computer-readable storage medium storing at least one software code portion, the software code portion, when executed or processed by a computer, is configured to perform one or more of the method steps as described above.

The invention will be further illustrated with reference to the attached drawings, which schematically will show embodiments according to the invention. It will be understood that the invention is not in any way restricted to these specific embodiments.

### BRIEF DESCRIPTION OF THE DRAWINGS

The following description of the figures of specific embodiments of the invention is merely exemplary in nature and is not intended to limit the present teachings, their application or uses.

FIG. 1 schematically depicts an endoscopic system enhanced with a coupling device to provide coherent light imaging according to an embodiment of the invention;

FIGS. 2A and 2B schematically depict a coupling device according to an embodiment of the invention;

FIG. 3 schematically depicts a coupling device according to an embodiment of the invention;

FIGS. 4A and 4B schematically depict a coupling device according to an embodiment of the invention;

FIG. 5A through 5D schematically depict optical switches according to embodiments of the invention;

FIG. 6A through 6C schematically depict optical switches according to embodiments of the invention;

FIGS. 7A and 7B schematically depict connections between a light source unit of an endoscopic system and a coupling device according to an embodiment of the invention;

FIG. 8 schematically depicts a system for adding coherent light imaging capability to an endoscopic system according to an embodiment of the invention; and

FIG. 9 depicts a method for generating a coherent light image according to an embodiment of the invention.

### DETAILED DESCRIPTION

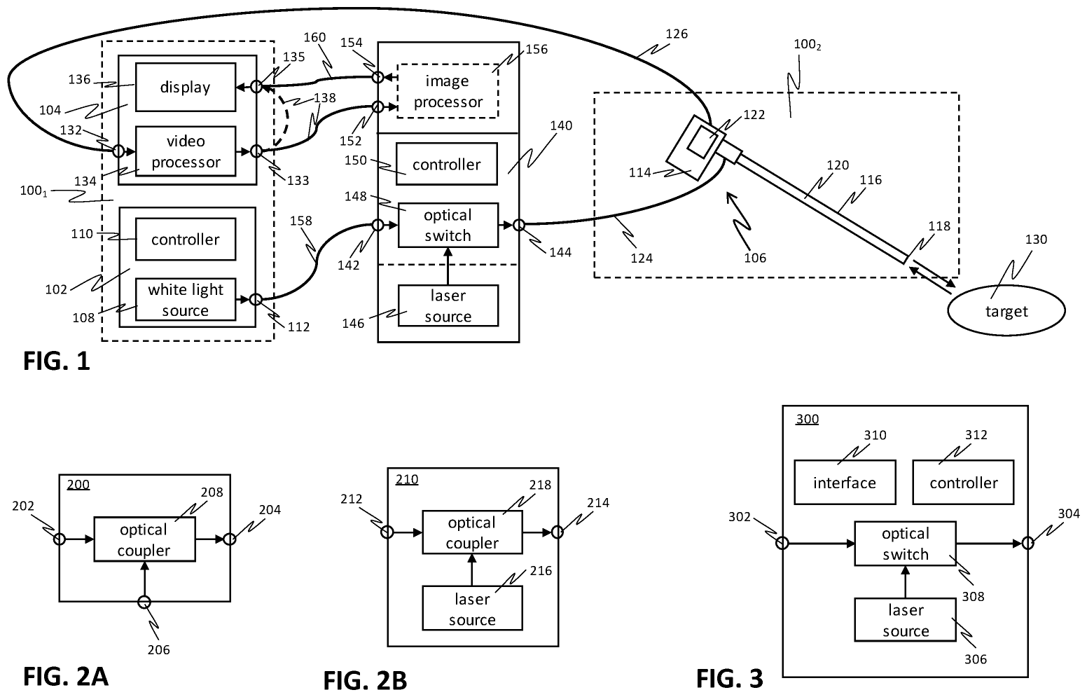

FIG. 1 schematically depicts an endoscopic system enhanced with a device to provide coherent light imaging according to an embodiment of the invention. The endoscopic system 100₁,100₂ may comprise a light source unit 102, a display unit 104, and an endoscope 106, e.g., a laparoscope.

The light source unit 102 may comprise a light source 108 for generating incoherent light, e.g., a white light source. The light source unit may further comprise a controller 110 for controlling the light source, e.g., controlling the light intensity or the spectral qualities of the light source. The controller may be configured to receive input from a user and/or from other system components. The light source unit may further comprise an optical output connector 112 for connecting an optical input, e.g., light guide 124, of the endoscope 106.

The endoscope 106 may comprise a handle 114 for operating the endoscope and a insertion tube 116. The insertion tube may have a proximal end connected to the handle and a distal end with a tip 118 for being inserted into a patient. The insertion tube may be a rigid tube or shaft or a flexible tube.

The endoscope may further comprise an optical input for receiving light via an input light guide 124, e.g., an optic fiber bundle. The light input may be connectable to the handle or to the insertion tube.

The insertion tube may comprise a light guide 120 for guiding light from the optical input to the tip 118 for illuminating a target area 130. The optical components between the optical output 112 of the light source unit and the tip of the endoscope may collectively be referred to as a light delivery system of the endoscopic system.

The endoscope may further comprise an image sensor 116, e.g., an RGB camera, for imaging the target area. In some endoscopes, the image sensor may be placed at the tip of the endoscope; such systems may also be referred to as chip-on-the-tip systems. In other endoscopes, the image sensor may be placed in the handle and the insertion tube may comprise an optical guide, e.g., a rod lens, for guiding light reflected or emitted by the target area to the optical sensor.

In some endoscopes, the handle 114 and the insertion tube 116 may be detachable. Thus, it may be possible to exchange the image sensor 122 for a different type of image sensor, while retaining the same insertion tube 116.

The endoscope may be connectable to a video input connector 132 of the display unit 104 using a video signal cable 126. The endoscope may further comprise wired or wireless connections for e.g., remotely controlling the light source unit 102 and/or the display unit 104. For example, the video signal cable may also be a data cable allowing bidirectional data transport. The endoscope may further comprise a power source or a power connector for receiving power to operate the image sensor.

The display unit 104 may comprise a video input connector 132 for receiving video input from the endoscope 106. The display unit may further comprise a video processor 134 for processing the video input signal, and a display 136 for displaying video or images received from the endoscope. The display may receive video input from the video processor via an (external) video cable 138, connecting a video output connector 133 of the video processor with a display input connector 135. In other embodiments, the display may receive input from the video processor via an internal connection.

The display unit 104 may further comprise a controller configured to receive input from a user and/or from other system components, e.g., for pausing the video or taking snapshots. The display unit may also provide output to other system components; for example, the display unit may communicate with the light source unit 102 to adjust the light intensity if the image in the video input signal appear to be overexposed or underexposed.

The display unit may further comprise or be connectable to a storage device for storing e.g., image data. In other endoscopic systems, the display may be separate device from the coupling device comprising the video processor. In some endoscopic systems, the video processor and the light source may be included in a single device.

Thus, the endoscopic system 100₁,100₂ described so far may be a (commercially available) endoscopic system for imaging a target area 130 in a patient body using incoherent light, e.g., white light. Certain embodiments in this disclosure relate to a device that allows to use the same, or essentially the same, endoscopic system for imaging the target area using coherent light, e.g., laser light together with incoherent light imaging or alternating with incoherent light imaging. Thus, new capabilities, in particular imaging capabilities such as fluorescence imaging and/or laser speckle contrast imaging, may be added to an existing endoscopic system, without having to exchange the light source or, at least, the insertion tube of the endoscope.

Therefore, a coupling device 140 for coupling coherent light into the light delivery system of the endoscopic system may be inserted between the optical output 112 of the light source unit and the optical input of the endoscope. The coupling device may comprise an optical input 142 for receiving the incoherent light from the endoscopic system and an optical output 144 for providing the incoherent light to the light delivery system. The optical input may comprise an optical input connector for releasably connecting a light guide 158. Similarly, The optical output may comprise an optical output connector for releasably connecting a light guide 124.

In the depicted embodiment, the coupling device's optical input 142 may be connectable to the optical output 112 of the endoscopic system's light source unit 102 via a light guide 158, e.g., a fiber bundle. The coupling device's optical output 144 may be connectable to an optical input of the endoscope via the light guide 124. In a set-up without the coupling device, light guide 124 might be connected directly to the light source unit's optical output 112. A releasable connection to connect and disconnect a light guide to a light source is a standard feature for endoscopic systems.

The coupling device 140 may further comprise a first light source 146, e.g., a laser, for generating coherent light of a first wavelength. In a different embodiment, the laser source might be a separate device and the coupling device may comprise a coherent light input for receiving coherent light of the first wavelength from a separate laser source. The coupling device may further comprise an optical switch 148 for selectively coupling the coherent light of the first wavelength to the optical output 144 and hence, when the light guide 124 is connected, into the light guide 124. The coupling device may also comprise a controller for controlling the optical switch.

In an embodiment, the coupling device 140 may further comprise a video signal input connector 152 for receiving a video input signal. The video signal may be preprocessed by the video processor 134 or may be obtained directly from the image sensor 122 in the endoscope. The coupling device may also comprise a video signal output connector 154 for providing a video output signal. The coupling device may further comprise an image processing module 156 for processing the video signal. Dedicated image processing software may take advantage of the possibilities of coherent light imaging, e.g., by implementing laser speckle contrast imaging. In a different embodiment, the image processing module and the video signal input and output connectors may be embodied in a separate device.

In the depicted embodiment, the coupling device's video signal input connector 152 may be connectable to a video signal output of the display unit 104 via a signal cable 138. In other embodiments, the coupling device's video signal input connector may be connectable directly to a video signal output of the endoscope 106. In some such embodiments, the signal cable may allow bidirectional data transport and may for example send trigger or control signals from the endoscope to the coupling device or vice versa. The coupling device's video signal output connector 154 may be connectable to the display input connector 135 of the display unit 104 via the signal cable 160. In a set-up without the coupling device, signal cable 138 might be connected directly to the display input connector 135.

FIGS. 2A and 2B schematically depict a coupling device according to an embodiment of the invention. In particular, FIG. 2A depicts a very basic embodiment of a coupling device 200 for coupling coherent light into a light delivery system of an endoscopic system. The depicted coupling device comprises an optical input 202 for receiving incoherent light, e.g., white light, from a light source of the endoscopic system and an optical output connector 204 for connecting to a light delivery system of the endoscopic system. The coupling device further comprises a coherent light input 206 for receiving coherent light of a first wavelength. The coherent light input optionally comprises a collimator to prevent or reduce divergence of the coherent light bundle. The coupling device further comprises an optical coupler 208 for receiving the incoherent light and the coherent light and for providing a coupled light beam. The coupled light beam may comprise the coherent light and the incoherent light simultaneously and/or alternately. The coupling device is further configured for injecting the coupled light beam into the optical output 204.

Thus, the coupling device may be configurable in a first state and a second state, which may partially or completely overlap. In the first state, the coupling device may comprise an unobstructed incoherent light beam path for propagating the incoherent light from the optical input to the optical output. In the second state, the coupling device may comprise an unobstructed coherent light beam path for propagating the coherent light from the coherent light source or the coherent light input to the optical output. The optical coupler may couple or combine the incoherent light beam path and the coherent light beam path onto a shared optical path configured to, simultaneously and/or alternately, inject the incoherent light and the coherent light into the optical output and, hence, if present, into a light guide connected to the optical output connector.

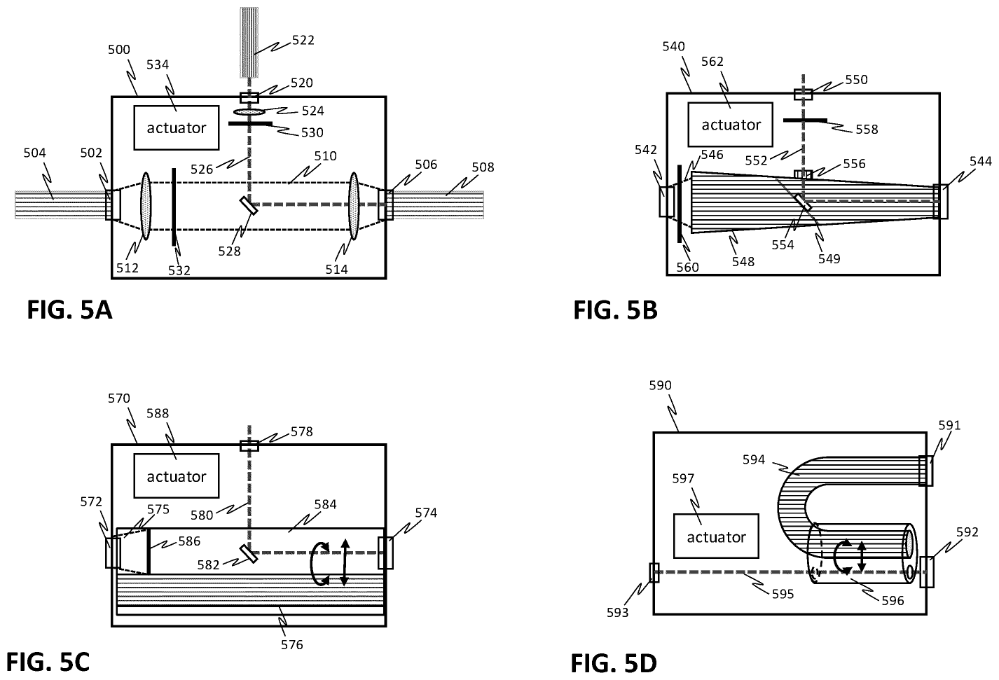

The optical coupler 208 may be a static optical coupler which combines the incoherent light and the coherent light simultaneously on a single optical path, for example a Y-cable joining fibers connected to the incoherent light input and fibers connected to coherent light input into a single fiber bundle. However, due to the light loss associated with an Y-cable, in particular as regards the incoherent light, such an embodiment is not preferred. Thus, the optical output 204 may provide both the incoherent light and the coherent light simultaneously to the light delivery system. Alternatively, the optical coupler 208 may be an optical switch for selectively coupling the coherent light into the optical output 204. Thus, the optical output may provide either only the incoherent light or, depending on the optical switch, only the coherent light and/or both the incoherent light and the coherent light. Embodiments of the optical coupler are discussed in more detail with reference to FIG. 5A-D.

FIG. 2B depicts a similar coupling device 210 comprising an optical input 212, an optical output 214, and an optical coupler 218 as described above with reference to FIG. 2A. The coupling device 210 further comprises a first coherent light source 216 for generating coherent light of a first wavelength.

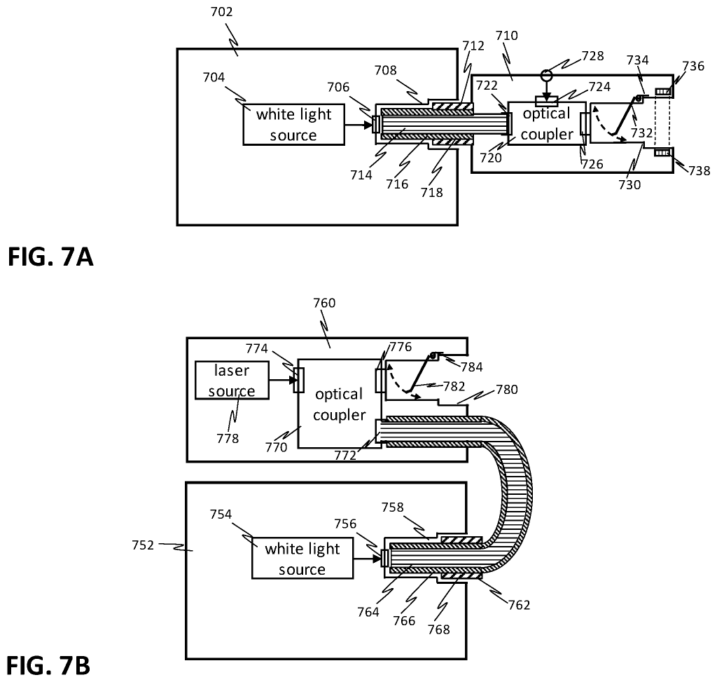

Using a coherent light input configured to receive coherent light from an external coherent light source as in FIG. 2A may result in a small coupling device which may be placed in a mostly unrestricted way in an endoscopy system. For example, in some embodiments, the coupling device might be inserted directly into the optical output of the endoscopic system's light source unit. This may result in a very low white light loss, as will be discussed below with reference to FIG. 7.

Additionally, using a coherent light input allows for different laser sources to be connected, which may be selected based on different imaging needs.

On the other hand, when the coupling device comprises an (internal) first coherent light source as in FIG. 2B, the coherent light may propagate through free space to the optical output, or at least to the optical coupler. This may result in higher quality coherent light (e.g., higher power, smaller spectral bandwidth and/or a longer coherence length), as there is no need to use optical components such as optical fibers which may be detrimental to the quality of the coherent light. It is typically not possible to have an external coherent light source and free space coherent light propagation because of safety issues and alignment difficulties.

FIG. 3 schematically depicts a coupling device according to an embodiment of the invention. In particular, FIG. 3 depicts a coupling device 300 comprising an optical input 302, an optical output 304, and a first coherent light source 306 for generating coherent light of a first wavelength as discussed above with reference to FIGS. 2A and 2B. The coupling device also comprises an optical switch 308 for selectively coupling the coherent light into the optical output 304.

The coupling device 300 may further comprise an interface 310 for operating the optical switch 308. The interface may comprise a physical trigger on the coupling device, e.g., a button, or a software-based trigger, e.g., a function (virtual button) on a touch screen. In other embodiments, the interface may comprise an input for receiving a wired or wireless trigger signal from an external device, for example from a part of the endoscopic system or from an external control device. A wireless trigger signal may use e.g., WiFi, Bluetooth, or an acoustical or optical communication protocol. Some endoscopic systems may be configured to provide a trigger signal when certain functionality of the endoscopic system is activated, e.g., a photo taking function. Such a trigger signal may be used to activate the optical switch. An external control device may be operable by an operator of the endoscopic system, e.g., a surgeon or endoscopist, and may comprise a foot switch or a remote control, preferably attachable to a handle of an endoscope, e.g., using an elastic band. The interface may also comprise e.g., a microphone and voice recognition software for voice-based operation of the optical switch. The interface may further provide feedback, preferably visual feedback, regarding the state of the optical switch.

The optical switch 308 may be configured for switching between a first state wherein the coherent light is not coupled the optical output 304, and a second state wherein the coherent light is coupled to the optical output. This way, an operator may select whether or not to use the laser light, without having to remove the coupling device.

In an embodiment, in the first state, the optical switch 308 may be configured to provide the incoherent light at the optical output 304. In the second state, the optical switch may be configured to not provide the incoherent light at the optical output. In many applications, a user may want to switch between only incoherent light and only coherent light.

In some embodiments, the optical switch 308 may also be configured for switching to a third state, in which third state the optical switch may be configured to provide both the coherent light and the incoherent light at the optical output 304.

The interface 310 may comprise a mechanical or electrical trigger. In case of a mechanical trigger, the switch may be manually operated by shifting between a first position, corresponding to the first state, and a second position, corresponding to the second state. In such an embodiment, the coupling device may not comprise a controller.

In a different embodiment, the coupling device 300 may further comprise a controller 312 for controlling the optical switch. In such an embodiment, the interface may send a trigger signal to the controller, and the controller operates the optical switch. In a typical embodiment, the optical switch is an electrically operated switch with a moving mechanical part actuated with a motor which is controlled by the controller.

In some embodiments, the controller may control the optical switch to switch between the first state and the second state independent of a trigger signal, e.g., at fixed time intervals. In such an embodiment, the coupling device need not comprise an interface.

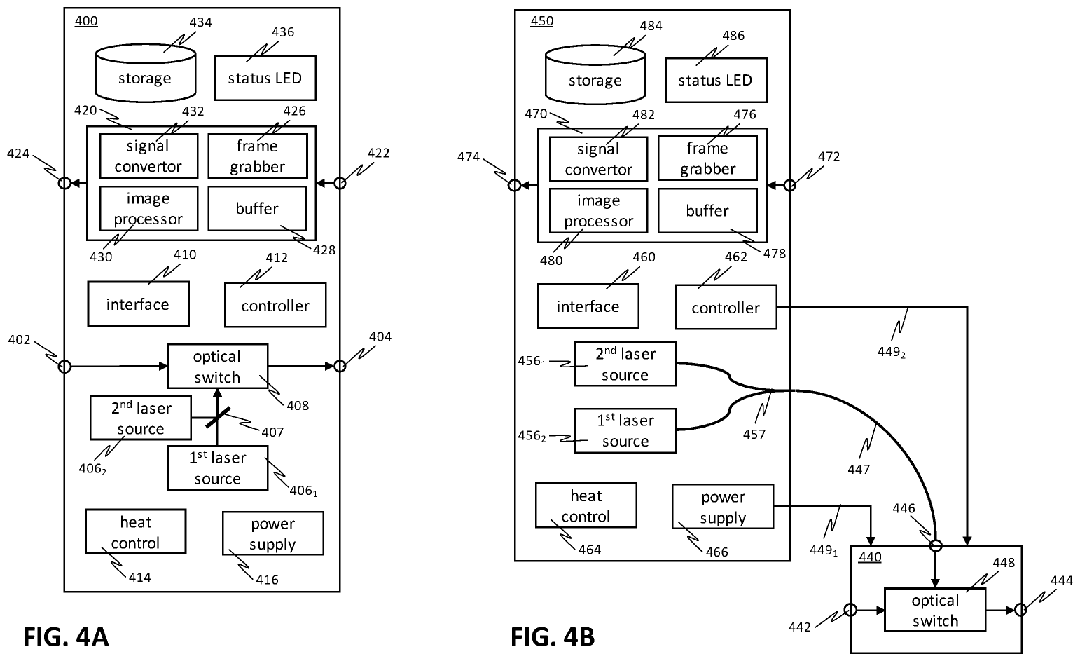

FIGS. 4A and 4B schematically depict a coupling device according to an embodiment of the invention. In particular, FIG. 4A depicts a coupling device 400 comprising an optical input 402, an optical output 404, an optical switch 408, an interface 410, and a controller 412 as discussed above with reference to FIGS. 2A, 2B, and 3.

The coupling device further comprises a first coherent light source 406₁ for generating coherent light of a first wavelength, a second coherent light source 406₂ for generating coherent light of a second wavelength, and a laser coupler 407, e.g., a dichroic mirror or a polarizing beam splitter, for coupling the coherent light of the first wavelength with the coherent light of the second wavelength. Alternatively, fiber-based laser coupling as described hereunder with reference to FIG. 4B may be used. Typically, the second wavelength is different from the first wavelength. By using a laser coupler, the coherent light of the first wavelength and the coherent light of the second wavelength may be combined on a single optical path.

A second coherent light source may be advantageous for laser speckle contrast images, as images obtained using the second wavelength may be used to correct images obtained using the first wavelength. A dichroic mirror is an efficient way to couple or combine the light of the second wavelength with the light of the first wavelength. In some embodiments, even more coherent light sources may be added in a similar way. This may be useful for e.g., three-dimensional laser speckle contrast imaging, based on laser speckle contrast images obtained with coherent light of a plurality of different wavelengths.

The coupling device 400 may further comprise an image processing module 420. The image processing module may receive a video signal from a video input connector 422 and provide a video signal to a video output connector 424. Typically, an endoscope may be connected to the video signal input connector, either directly or indirectly via, e.g., a video processor of the endoscopic system, and a display unit may be connected to the video signal output connector.

The video signal may be an analogue signal or a digital signal. The video processing module 420 may comprise a frame grabber 426 for converting a video signal into separate image suitable for further processing. The video processing module may comprise a buffer 428 for temporarily storing one or more images. The one or more images may then be processed by an image processor 430. The image processor may comprise a computer-readable storage medium having computer-readable program code embodied therewith, and a processor, preferably a microprocessor, coupled to the computer-readable storage medium.

The image processor may be configured to compute e.g., laser speckle contrast images or fluorescence images. For example, the image processor may be configured to execute a LSCI-based clinical perfusion imaging scheme as described in the review article by W. Heeman et al, ‘Clinical applications of laser speckle contrast imaging: a review’, J. Biomed. Opt. 24:8 (2019), which is hereby incorporated by reference. Additionally or alternatively, the image processor may be configured to execute a fluorescence-based clinical perfusion imaging scheme, preferably ICG-based fluorescence imaging, as described in the review article by A. V. DSouza et al, ‘Review of fluorescence guided surgery systems: identification of key performance capabilities beyond indocyanine green imaging’, J. Biomed. Opt. 21:8 (2016), which is hereby incorporated by reference.

The video processing module 420 may further comprise a signal convertor 426 for converting the output generated by the image processor into an output video signal compatible with the input video signal. For instance, if the video processing module receives an SDI signal from the video input and the image processor outputs a HDMI signal, the image convertor may be an HDMI to SDI convertor. Thus, the video output signal is compatible with the video input signal, ensuring that it may be processed by the display unit in the same way the signal from the endoscope would be.

In an embodiment, the coupling device may comprise a screen or be connectable to a different display. In that case, a signal convertor may not be needed. Likewise, if the image processor provides output of the same type as is received from the endoscope, a signal convertor may not be needed. In an embodiment, the coupling device may have more than one video output connector, e.g., a first output connector for outputting an unprocessed video signal, and a second output connector for outputting a processed signal.

In an embodiment, the video processing module 420 may communicate with the controller 412. The video processor module may then e.g., provide an unprocessed signal when the optical switch is in the first state, providing only incoherent light, e.g., white light, to the endoscope, and provide a processed signal when the optical switch is in the second state, providing coherent light to the endoscope. In an embodiment, the video processing module and the controller may be implemented as software modules on the same hardware module.

The coupling device 400 may further comprise a storage 434 for storing images, or a storage output connector to connect to an external storage device. The coupling device may, for instance, be configured to store all image data, to store only processed and/or unprocessed image data when the optical switch is in the second state, of only when a storage signal is received from the controller 412.

The coupling device 400 may also comprise a power supply 416 for providing power to electrical components, e.g., the one or more laser sources 406₁₋₂, the optical switch 408, the controller 412, the video processing module 420, and any other components requiring power. One or more heat control units 414, which may comprise e.g., heat sinks and/or fans, may control a temperature of one or more components such as the laser sources and the video processing module. One or more status LEDs 436 may indicate the status of various components, e.g., power being supplied by the power supply, the state of the optical switch, the optical output connector being engaged, et cetera. A housing may protect the components.

FIG. 4B schematically depicts a system comprising a coupling device 440 and a processing device 450. The coupling device 440 may comprise an optical input 442, an optical output 444, and an optical switch 448 as described with reference to FIG. 4A. The processing device 450 may comprise an interface 460, a controller 462, a heat control 464, a power supply 466, a video input connector 472, a video output connector 474, storage means 484, one or more status LEDs 486, and a video processing module 470 comprising a frame grabber, a buffer, an image processor and a signal convertor, also as described with reference to FIG. 4A.

The processing device 450 may further comprise a first coherent light source 456₁, a second coherent light source 456₂, and a laser coupler 457. In the depicted example, the laser coupler comprises a fiber coupler. As the coherent light is guided from the processing device 450 to the coupling device 440 using an optical fiber 447, this is an efficient way of combining the plurality of laser sources. In other embodiments, the first coherent light and the second coherent light might first be combined using a laser coupler as explained with reference to feature 407 in FIG. 4A, and the coupled or combined coherent light bundle might subsequently be injected into a fiber.

The optical fiber 447 may be connectable to a coherent light input 446 of the coupling device 440. Optionally, the coherent light input may comprise a collimator. The coupling device may further be connectable to the processing device 450 by one or more cables 449₁₋₂ for receiving power and control signals to operate the optical switch 448.

FIG. 5A through 5D schematically depict optical switches according to embodiments of the invention. Some of the embodiments described below with reference to FIG. 5A through 5D may be implemented as non-switching optical couplers. These embodiments are typically more light-efficient than e.g., a Y-cable coupler as described above with reference to FIG. 2A.

In particular, FIG. 5A depicts an optical switch 500 comprising an incoherent light input 502, a coherent light input 520, and a light output 506. The incoherent light input may be configured to receive incoherent light via a first light guide 504, e.g., a light pipe or a fiber bundle. The incoherent light input may be an optical input of a coupling device or may be optically connected to the optical input. The light output may be an optical output of the coupling device or may be optically connected to the optical output. Thus, the light output may provide light to a second light guide 508, which may be an internal light guide or a light guide of an endoscopic system.

The optical switch 500 may further comprise a first convergent lens 512 to reduce divergence of the incoherent light 510 from the coherent light input 502 and a second convergent lens 514 to focus the incoherent light on the optical output 506. In an alternative embodiment, one or more convergent lenses may be replaced by one or more concave mirrors. Concave mirrors may have superior optical qualities than lenses, e.g., less light absorption and/or less chromatic aberration; however, lenses may be cheaper than concave mirrors. It is evident to the skilled person that when concave mirrors are used, the position of the incoherent light input and, optionally, of the coherent light input, may be adjusted accordingly.

The optical switch 500 may further be configured to receive coherent light from a coherent light source 522 or via an third light guide, e.g., an optical fiber. If the coherent light propagates through free space, the coherent light input 520 may be simply a hole or a window in a housing of the optical switch. In some embodiments, in particular when the coherent light input receives coherent light via a third light guide, the optical switch may comprise a third convergent lens 524 to reduce divergence of coherent light bundle 526.

The optical switch 500 may further comprise a mirror 528, positioned between the first convergent lens 512 and the second convergent lens 514. The mirror may be configured to steer the coherent light beam 526 onto the light output 506. As the coherent light bundle is much smaller in diameter than the incoherent light bundle 510, the mirror may be small relative to the diameter of the incoherent light bundle. The mirror may be mounted using a small, optionally transparent, holder. Thus the intensity loss of the incoherent light may be small. As both the incoherent light 510 and the coherent light 526 travel through free space between the light inputs 502,520 and the light output 506, intensity loss is further minimized.

The optical switch 500 may further comprise a first shutter 530, preferably a rotational shutter, positioned between the coherent light input 520 and the mirror 528. The first shutter may be configured to move between at first state blocking the coherent light bundle 526 and a second state not blocking the coherent light bundle. By closing and opening the first shutter, the optical switch may switch between a first state in which the coherent light is not couple to the light output, and a second state in which the coherent light is coupled to the light output.

In an alternative embodiment, the first shutter 530 may be a non-mechanical light blocking mechanism e.g., a liquid crystal light valve, an acousto-optical modulator, an electro-optical modulator such as a Pockels cell, or a photo-elastical modulator. In some cases, the coherent light may need to be polarized, e.g., by using a polarized laser source or by inserting a polarizer between the coherent light source and the first shutter.

In another embodiment, the coherent light beam 526 may be steered onto or away from the optical output 506 by moving mirror 528. In such an embodiment, the first shutter 530 may be removed. Alternatively, the mirror may be a switchable mirror, e.g., an electro-optically switchable mirror. The switchable mirror may be switched between a transparent state in the first state of the optical switch, and a reflective state in the second state of the optical switch. Depending on the size of the mirror, the movable or switchable mirror may also be used to block the incoherent light in the second state, thus removing the need for the second shutter 532.

By selecting a suitable placement for the incoherent light input and the coherent light input, a single mirror may be used to reflect either the incoherent light or the coherent light onto the optical output.

In yet another embodiment, the coherent light may be switched on and off by switching on and off a coherent light source. This way, there is no need to include a moving part. However, using a shutter may be faster than switching the coherent light source.

Optionally, the optical switch 500 may comprise a second shutter 532, preferably a rotational shutter, positioned between the incoherent light input 502 and the mirror 528. The second shutter may be configured to move between a first state not blocking the incoherent light bundle 510 and a second state blocking the incoherent light bundle. Alternatively, the second shutter may be implemented as a liquid crystal light valve.

The first shutter 530 and, optionally, the second shutter 532 may be moved by one or more actuators 534.

In an embodiment, the first shutter 530 and the second shutter 532 may be operated jointly, such that when the first shutter blocks the coherent light bundle 526, the second shutter does not block the incoherent light bundle 510, and vice versa. In another embodiment, the first and second shutters may be operated independently, allowing the incoherent light and the coherent light to be used either separately or combined.

The first shutter 530 and the second shutter 532 may be rotating shutters, allowing fast switching between the first state and the second state. By using rotating shutters in counter phase, alternating illumination with incoherent light and coherent light may be obtained at a relatively high frame rate.

Thus, the optical switch 500 may be configured to switch automatically back and forth between the first state and the second state, preferably with a frequency of at least 10 Hz, more preferably with a frequency of at least 30 Hz, even more preferably with a frequency of at least 100 Hz. By alternating between two illumination states, alternating images may be acquired. This may be beneficial for further processing, where e.g., images based on coherent light imaging may be overlaid on or shown side to side with the preceding or subsequent incoherent light image, thus combining information from two imaging modalities.

In a different embodiment, a static optical coupler could be obtained by removing the shutters 530,532 and the actuator 534.

In another embodiment, the positions of the incoherent light input 502 and the coherent light input 520 may be exchanged, and mirror 528 may be configured to reflect incoherent light from the incoherent light input to the light output 506. The mirror may further comprise a hole or window to allow the coherent light bundle to propagate from the coherent light input to the light output.

In yet another embodiment, both the incoherent light input 502 and the coherent light input 520 may be placed under an angle with respect to the light output 506, and the mirror 528 may comprise two or more reflecting surfaces for reflecting coherent light and/or incoherent light onto the light output.

In yet another embodiment, the coherent light input may be placed close to the incoherent light input, e.g., both on a wall opposite the light output, and the coherent light and/or the incoherent light may be arranged to inject light under a small angle into the light output. In such an embodiment, the mirror may be removed. However, embodiments including a mirror may be easier to align and may have a less light loss, as both the coherent light and the incoherent light may be injected into the optical output under an optimal angle.

FIG. 5B depicts an optical switch 540 comprising an incoherent light input 542, a coherent light input 550, and a light output 544 as described with reference to FIG. 5A. The optical switch 540 may further comprise a first shutter 558, a second shutter 560, and an actuator 562 as described with reference to FIG. 5A.

However, instead of lenses 512 and 514 as depicted in FIG. 5A, the optical switch 540 may comprise a tapered light pipe 548 having a wide end for receiving incoherent light 546 from the incoherent light input 542 and a narrow end for providing the incoherent light and/or the coherent light to the light output 544. The incoherent light bundle exiting the incoherent light input is typically strongly divergent. The optimum width of the wide end is therefore dependent on the distance between the incoherent light input and the wide end.

The tapered light pipe 548 may be divided into a first part and a second part by a plane 549 intersecting the tapered light pipe between the wide end and the narrow end, a normal of the plane defining a non-zero angle with a longitudinal axis of the tapered light pipe. The optical switch may further comprise a mirror 554, positioned on the plane and configured to steer a coherent light beam 552 on the light output 544. The tapered light pipe may comprise a protrusion 556 having a surface normal to the coherent light path for minimized light reflection by the surface of the tapered light pipe.

Using a tapered light pipe instead of lenses may minimized chromatic aberrations, in particular of the incoherent light.

FIG. 5C depicts an optical switch 570 comprising an incoherent light input 572, a coherent light input 578, and a light output 574 as described with reference to FIG. 5A.

The optical switch 570 may further comprise a switching body 584 which may be moved between a first position corresponding to a first state of the optical switch 570 and a second position corresponding to a second state of the optical switch. In the first state, the optical switch may be configured to provide only incoherent light at the light output 574, while in the second state, the optical switch may be configured to provide only coherent light at the light output. The figure depicts the optical switch in the second state.

The switching body 584 may comprise a light guide 576, e.g., a light pipe, configured to guide the incoherent light from the incoherent light input 572 to the light output 574 when the switching body is in the first position. In the second position, the incoherent light 575 may be blocked by a shutter 586. Thus, in the second position, the switching body is configured not to guide the incoherent light to the optical output.

The switching body 584 may further comprise a mirror 582 configured to steer a coherent light beam 580 on the light output 574 when the switching body is in the second position. The mirror may further be configured not to steer the coherent light beam on the light output when the switching body is in the second position.

The switching body 584 may be moved between the first position and the second position by an actuator 588.

In an embodiment, the optical switch 570 may comprise a first end stop configured to stop the switching body 584 in the first position corresponding to the first state; and a second end stop configured to stop the switching body in the second position corresponding to the second state. The optical switch may further comprise a spring configured to keep the switching body in the first or second position if the switch is not being operated. This configuration is reliable, accurate, and relatively cheap.

Alternatively, a servomotor may be used for moving the switching body 584 between the first position and the second position. In that case, end stops may not be needed. Using a servomotor may be faster and more silent than using a (normal) motor and end stops.

In yet another alternative, the switching body 584 may be moved manually, using e.g., a lever. This is a cheap, but relatively slow and potentially less accurate way of switching.

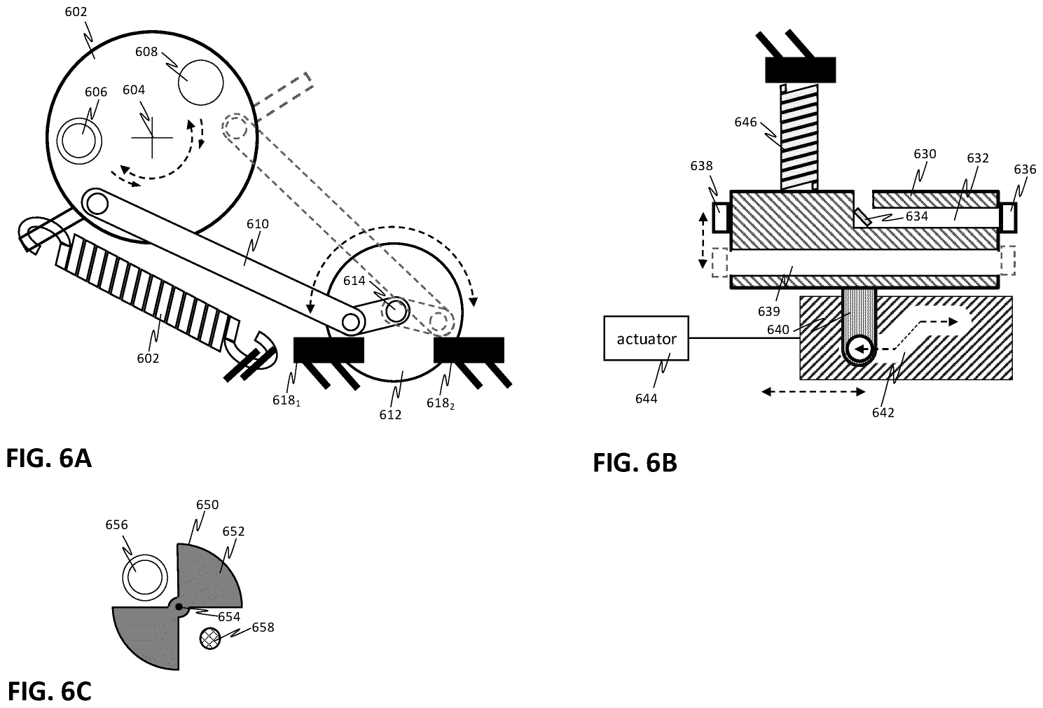

The switching body 584 may be moved between the first and second positions by e.g., a translating or rotating movement. In an embodiment, motion may be transferred to the switching body via a cam or via a crank. This is discussed in more detail below with reference to FIGS. 6A and 6B.

The use of light guide 576 introduces little light loss and minimizes chromatic aberrations. Alternatively, a system with lenses as depicted in FIG. 5A may be used.

FIG. 5D depicts an optical switch 590 comprising an incoherent light input 591, a coherent light input 593, and a light output 592 as described with reference to FIG. 5A.

The optical switch 590 may further comprise a switching body 596, configured to be moved between a first position and a second position. The switching body may comprise at least an end of a flexible light guide 594, e.g., a fiber bundle, a fiber bundle with fused ends, or a liquid light guide. In the first position, the switching body may be configured to guide incoherent light from the incoherent light input to the light output via the flexible light guide. The flexible light guide may extend beyond the incoherent light input and may e.g., be connectable directly to an optical output of an endoscopic system's light source unit. An advantage of this configuration is that coupling device may have an optical input and an optical output on the same side, e.g., the front side, which facilitates operation of the coupling device.

In the second position, the switching body 596 may be configured to provide coherent light 595 to the light output 592. In the depicted example, the beam path from the coherent light input 593 to the light output 592 may be a path through free space. In the second position, the coherent light beam path to the optical output may be unobstructed. The switching body may have a hole for letting through the coherent light. Alternatively, the switching body may have a size and shape such that in the second position, no part of it covers the path between the coherent light input and the light output.

This way, very few optical components are used to provide the coherent light at the optical output, which is beneficial for the spectrum and coherence length of the coherent light.

In a different embodiment, an optical fiber may be used to guide coherent light from the coherent light input 593 to the light output 592. This may further increase the flexibility of the setup, and may make the coupling device less sensitive to misalignment of components.

The optical switch 590 may further comprise an actuator 597, end stops, a spring, et cetera, as described with reference to FIG. 5C.

FIG. 6A through and 6C schematically depict optical switches according to embodiments of the invention. In particular, FIG. 6A depicts a driving mechanism for a switching body 602 of an optical switch. The switching body may be configured to rotate around an axis 604. As discussed in more detail above with reference to FIGS. 5C and 5D, the switching body may comprise a first light guide 606, e.g., a light pipe, a fiber bundle, or a liquid light guide, configured to guide incoherent light from an incoherent light input to a light output of the optical switch when the switching body is in a first position (depicted). The switching body may further comprise a second light guide 608, e.g., a hole or an optical fiber, configured to guide coherent light from a coherent light input to the light output of the optical switch when the switching body is in a second position (indicated with dashed lines). When the coherent light propagates through free space, the switching body may be shaped such that it does not block the coherent light beam in the second position, and does not need to comprise a hole.

The switching body 602 may be connected to a motor 612 via a crank 610. One end of the crank may be rotatably connected to the switching body 602. Another end of the crank may be rotatably connected to an off-axis connection point of the motor. In the first position, the crank may be pulled against a first end stop 618₁ by a spring 620, in this embodiment attached to the switching body. In other embodiments, the spring may be attached to a different part. The spring may also push instead of pull. The end stop and spring ensure the switching body is accurately positioned in the first position. In different embodiments, the spring and, optionally, the end stop may be left out.

When the motor receives 612 a switching signal, the motor may rotate around axis 614 to a second position. In the second position, a part of the crank or another suitable component may be pulled against a second end stop 618₂ by spring 620.

In an embodiment, a servomotor may be used to drive the switching body. Servomotors may be very accurate, and therefore, the end stops 618₁₋₂ and the spring 620 may be excluded.

In an embodiment, the switching body 602 may be attached directly to an axis 604 of a motor, e.g., a servomotor.

FIG. 6B schematically depicts a driving mechanism for moving a switching body 630 of an optical switch between a first position and a second position using a cam. Although the depicted switching body corresponds to an optical switch as depicted in FIG. 5C, the same driving mechanism may be used to move any suitable switching body, e.g., one as depicted in FIG. 5D.

The switching body 630 may comprise a first light path 632 for guiding light from a coherent light input to a light output 636 when the switching body is in the second position (depicted). The light path may comprise e.g., a mirror 634. Alternatively, the light path may comprise e.g., an optical fiber. In the second position, part of the switching body may block light coming from an incoherent light input 638. The switching body may further comprise a light guide for guiding incoherent light from the incoherent light input to the light input when the switching body is in the second position (the relative position of the incoherent light input and the light input with respect to the light guide has been shown using dashes).

A shaft 640 may be connected to the switching body 630. An actuator 644 may be configured for moving cam 642. By moving the cam in a first direction, e.g., parallel to a longitudinal axis of the switching body, the switching body may be moved in a second direction, e.g., a lateral direction perpendicular to the first direction. The cam may be shaped with high accuracy, thus ensuring accurate positioning of the switching body. The positioning may further be improved by a spring 646, in particular in the first state.

FIG. 6C schematically depicts part of an optical switch comprising a rotating shutter. The shutter 650 may comprise one or more shutter blades 652 attached to a rotatable axis 654. The shutter may be placed in the optical switch as described above with reference to FIGS. 5A and 5B. When the shutter rotates around the axis, the shutter blades may alternately obstruct and leave unobstructed an incoherent light or coherent light optical beam path 656.

The optical switch may further comprise a light sensor 658 positioned on one side of the shutter and a light emitter positioned on the other side of the shutter, together forming an optical sensor arrangement substantially parallel with the rotation axis. In the depicted embodiment, the optical sensor arrangement is configured such that the optical sensor may receive light from the light emitter when the optical beam path is not obstructed by the shutter blades and does not receive light when the optical beam path is obstructed by the shutter blades. Thus, the light sensor may provide a signal associated with the beam path being obstructed or unobstructed to a controller of the optical switch and/or to an interface.

Alternatively or additionally, an optical sensor arrangement may be positioned such that the optical sensor receives light when the optical beam path is obstructed and does not receive light when the optical beam path is unobstructed. Similar optical sensor arrangements may also be included in other embodiments, e.g., in the embodiments depicted in FIGS. 6A and 6B.

FIGS. 7A and 7B schematically depict connections between a light source unit of an endoscopic system and a coupling device according to an embodiment of the invention.

FIG. 7A depicts a light source unit 702 of an endoscopic system comprising an incoherent light source 704, e.g., a white light source, and an optical output 706 for providing light to an optical output connector 708. The optical output connector is shaped to receive a connector from a light delivery system. Different makes or brands may have differently shaped optical output connectors.

FIG. 7A further depicts a coupling device 710 for injecting coherent light into the light delivery system of the endoscopic system. The coupling device comprises an optical input connector 712 for connecting to the optical output connector 708 of the light source unit 702. In the depicted embodiment, the optical input connector comprises a rigid light pipe 714 for guiding light from the light source unit's optical output 706 to an optical coupler 720. The optical input connector may further comprise a shaft 716, preferably an opaque shaft, for protecting the light guide and preventing unwanted light from entering the light pipe. The shaft may cover the entire length of the light guide or parts of it, e.g., only exposed parts. The optical input connector may also comprise a holder part 718 for securing the light pipe to the light source unit's optical output connector 708. The holder part may extend over a part of all of the length of the light pipe and/or of the connector to provide mechanical support and protection of the light pipe. A rigid light pipe allows for a connection with little intensity loss to the incoherent light and limited changes in spectrum.

Preferably, the shape and dimensions of the optical input connector 712 are selected to be compatible with a predetermined type of optical output connector 708. In some embodiments, the optical input connector may be exchangeable or adjustable in order for the coupling device 710 to be compatible with a plurality of optical output connector types.

The optical coupler 720 may comprise an incoherent light input 722 for receiving incoherent light from the optical input connector. The optical coupler may further comprise a coherent light input 724 and a light output 726. The coherent light input may receive light from a coherent light source directly or via an coherent light input connector 728. It is an advantage of receiving coherent light via a coherent light connector that the coupling device can be relatively small and lightweight, allowing for the coupling device to be attached to the front of the light source unit 702 also when the light source unit is mounted in an optical tower, without requiring e.g., a dedicated external support structure, and without becoming cumbersome to the user by extending far. The optical coupler may be an optical switch as discussed above with reference to e.g., FIG. 5A through 5D, preferably an optical switch as shown in FIG. 5A through 5C.

The coupling device 710 may further comprise an optical output connector 730 for connecting to and providing light to the endoscopic system's the light delivery system. Preferably, the optical output connector is shaped and dimensioned to be compatible with the same type of optical connector as the coupling device's optical input connector 712. This way, the coupling device may be connected to a light delivery system that is also connectable directly to the light source unit 702.

As the coupling device 710 provides coherent light at its optical output 726, the coupling device's optical output connector 730 may advantageously be equipped with one or more laser safety features 732-740. For example, the optical output connector may comprise a shutter 732 for blocking light, in particular coherent light, from exiting the optical output connector 730 if there is no light delivery system connected to the optical output connector 730. The shutter may be pushed to a shut position by e.g., a spring 734 when the output connector is empty, and may be pushed to an open position when an input connector is inserted. Alternatively or additionally, the optical output connector may comprise an optical sensor 736,738. The optical sensor may comprise a light source 736 and a sensor 738. The sensor may provide a first signal to a controller of the coupling device if more than a predetermined amount of light from the light source 736 is detected by the sensor 738, and a second signal if less than the predetermined amount is detected. When the controller receives the first signal, the controller may, depending on the embodiment, e.g., switch off the first and/or second laser sources, and/or block the coherent light path by switching to a state in which the coherent light is not injected to the optical output connector.

In the depicted embodiment, the light guide 714 from the optical input connector 712 connects directly to the optical coupler 720. An advantage of this configuration is its constructional simplicity and low loss in light intensity. However, in other embodiments, there may be intermediate structures between the optical input connector and the optical coupler. Similarly, the light output 726 of the optical coupler is connected directly to the optical output connector 730. This is again advantageous for the light intensity and quality of the output light. Again, in other embodiments, there may be intermediate structures to guide light from the light output 726 to the optical output connector 730.

FIG. 7B depicts an alternative type of connection between a light source unit 752 of an endoscopic system and a coupling device 760. The light source unit may comprise an incoherent light source 754, e.g., a white light source, an optical output 756 and an optical output connector 758 as discussed above with reference to FIG. 7A.

The coupling device 760 may comprise an optical input connector 762 comprising a shaft 766 and a holder 768 as discussed above with reference to FIG. 7A. The input connector also comprises a light guide 764, but in this embodiment, the light guide is a flexible light guide, e.g., a fiber bundle, a fiber bundle with fused ends, or a liquid light guide. This allows for flexible placement of the coupling device, e.g., on a different platform of an optical tower housing the light source unit. As a result, there are less restrictions to e.g., size and weight, compared to a situation where the coupling device is appended to a front of the light source unit, facilitating incorporation of one or more coherent light sources 778 in the coupling device. This has an advantageous effect on the light quality of the coherent light, as the coherent light can travel almost entirely through free space without safety concerns.

The coupling device 760 may further comprise an optical coupler 770 comprising a incoherent light input 772, a coherent light input 774 and a light output 776. The optical coupler may be an optical switch as discussed above with reference to e.g., FIG. 5A through 5D, preferably an optical switch as shown in FIG. 5D. If the optical coupler is an optical switch as shown in FIG. 5D, the flexible light guide 764 may be the same as the flexible light guide 594, thus minimizing transitions and maximizing light quality.

The coupling device 760 may further comprise an optical output connector 780 and safety features, e.g., a shutter 782 operated by a spring 784 and/or an optical sensor (not depicted), as discussed above with reference to FIG. 7A.

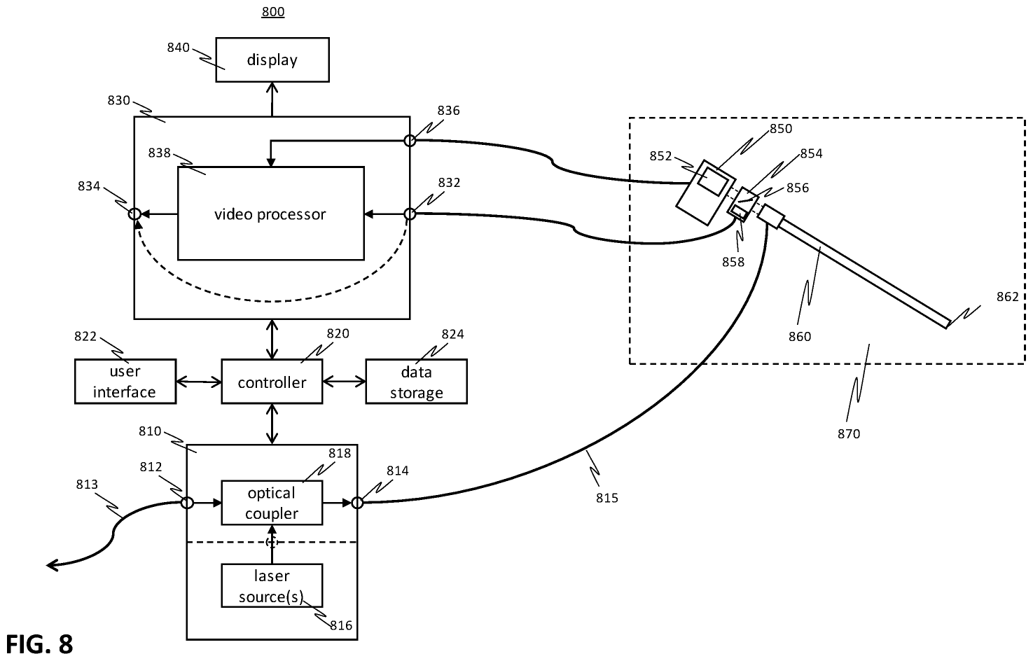

FIG. 8 schematically depicts a coherent light system for adding coherent light imaging capability to an endoscopic system according to an embodiment of the invention. The coherent light system 800 may comprise a coupling device 810 comprising an optical input 812 for receiving incoherent light from a light source of the endoscopic system via a light guide 813, an optical output 814 for providing light to a light guide 815 of a light delivery system of the endoscopic system, and a coherent light input for receiving coherent light of at least a first wavelength and/or one or more coherent light sources 816 for generating light of the at least first wavelength. The coupling device may further comprise an optical coupler 818 for simultaneously or alternately providing the incoherent light and the coherent light to the light delivery system.

Optionally, the coherent light system 800 may comprise a controller 820, operatively connectable to the optical coupler 818, for controlling the optical coupler. The controller may be included in the same device as the optical coupler and/or as the one or more laser sources, or may be embodied in a separate controller device and configured to communicate with the coupling device 810 using a wired or wireless connection.

Optionally, the coherent light system 800 may comprise a user interface 822 connectable to the controller 820 for receiving input for controlling the system and/or providing output regarding the status of the system. The user interface may comprise one or more buttons, e.g., a power button and a button for generating trigger signal. The user interface may also comprise one or more status lights. The user interface may be included in the same device as the controller and/or the optical coupler 818. Alternatively, the user interface may be embodied in a different device, e.g., a handheld device, configured to communicate via a wired or wireless connection with the controller and/or the coupling device 810. The user interface may also be distributed over several components of the system, e.g., a button attached to a handle of an endoscope 870 configured to send a trigger signal to the coupling device, and a status light on the coupling device indicating the status of the optical coupler 818. The status of the optical coupler is particular relevant when the optical coupler is an optical switch.

Optionally, the coherent light system 800 may comprise or be connectable to an internal or external data storage 824 for storing information related to the status of the coupling device and/or for storing images acquired with the endoscopic system, if the system comprises a video processing module 830. The data storage may be operatively connectable to the controller 820 via a wired or wireless connection.

Optionally, the coherent light system 800 may comprise a video processing module 830. The video processing module may comprise a first video input connector 832 for receiving a first video stream from a camera for capturing endoscopic images, the camera preferably being included in or attached to the endoscope 870. The video processing module may further comprise a video output connector 834 for providing the video input stream or a derivative thereof to a video processing module of the endoscopic system. In some embodiments, the video processing module may comprise a second video input connector 836 for receiving a second video stream from a camera for capturing endoscopic images. The video processing module may further comprise a video processor 838 for processing the first and, optionally, second video streams. Preferably, the video processor comprises a dedicated graphics processor for processing the video stream. The video processor may further comprise a general processing unit and a memory for storing software, e.g., image processing software for generating laser speckle contrast images and/or fluorescence images. The video processing module may further comprise e.g., a frame grabber, a video converter, and a buffer.

The image processor may comprise a computer-readable storage medium having computer-readable program code embodied therewith, and a processor, preferably a microprocessor, coupled to the computer-readable storage medium. The computer-readable program code may comprise instructions to execute method steps for determining a coherent light image, preferably a fluorescence image such as an ICG-based image or a laser speckle contrast image.

The video processing module may be implemented as a cloud-based module. In such an embodiment, the video processing module may comprise a network connection for connecting to the Internet, and the image processor may be an Internet-connected computing device.

In an embodiment, the video processor may process the first and/or second video streams in dependence on the status of the optical coupler. For example, if the optical coupler is an optical switch, the video processor may be configured or controlled by the controller 820 to only process the one or more video streams when the optical switch is in a state in which the coherent light is provided to the light delivery system.

The video processing module 830 may be included in the same device as the controller 820, or may be embodied in a separate image processing device and configured to communicate with the controller using a wired or wireless connection. The video processor and the controller may also be embodied as software modules running on shared hardware, e.g., an embedded computing module comprising a central processing unit (CPU), a memory, and, optionally, a graphics processing unit (GPU).

The coherent light system 800 may further comprise a display 840, connectable to the video processing module 830. The system may thus be configured to display images or video based on coherent light imaging on the display 840, while images or video based on incoherent light imaging are displayed on display included in the endoscopic system.

The endoscopic system may comprise an endoscope 870. The endoscope may comprise a flexible or rigid insertion tube 860 with a distal tip 862 for inserting into an object, preferably a patient, e.g., a body of a human or an animal, preferably a living body. Some endoscopic systems, typically systems with a flexible insertion tube, may have an image sensor mounted at or near the distal tip 862. Other endoscopic systems, typically systems with a rigid insertion tube, may have an imaging unit comprising an image sensor, e.g., an RGB camera, located in or near a handle of the endoscope. In some systems, the imaging unit is detachable from the insertion tube.

Optionally, the coherent light system 800 may comprise an imaging unit 850 configured to be attached to a proximal end of an insertion tube of an endoscope. The imaging unit may comprise a camera, e.g., an RGB or an RGB/IR camera 852. An advantage of an RGB/IR camera is that infrared coherent light may be acquired simultaneously with incoherent light images or white light image. This way, images obtained using coherent light and images based on the incoherent light may be combined in an relatively easy manner, without requiring e.g., alignment of the images based on coherent and incoherent light. If the first wavelength is in the visible spectrum, coherent light images may be obtained by reading the respective channels of the RGB camera, e.g., if the first wavelength is in the red part of the electromagnetic spectrum, a coherent light image may be obtained by reading or using only the red pixels of an RGB image captured during illumination of a target with red coherent light and preferably without simultaneous illumination with incoherent light.

Alternatively, the coherent light system 800 may comprise a dedicated imaging module 854 for acquiring images of light at the first wavelength. The dedicated imaging module may comprise a beam splitter 856, e.g., a dichroic mirror or polarizing beam splitter, for splitting light collected by the endoscope in a first light bundle, comprising predominantly light of approximately the first wavelength or light of a wavelength emitted by a fluorescent marker, and a second light bundle, comprising the remainder of the light. The dedicated imaging module may further comprise a dedicated image sensor 858 for imaging the first light bundle.

The dedicated imaging module 854 may be configured to be attached to a proximal end of the 860 insertion tube of the endoscope 870. The dedicated imaging module may further be configured to receive an imaging unit 850 that is configured to be attached to the proximal end of the insertion tube of the endoscope. This way, dedicated imaging, e.g., infrared imaging, may be added to an endoscope with a detachable camera. If the first wavelength is in the infrared part of the electromagnetic spectrum, an endoscope without an infrared filter in the insertion tube or connection point should be selected.

In some embodiments of the coherent light system 800, the optical coupler 818 may be configured as an optical switch capable of switching at a high frequency, e.g., 60 Hz. In such an embodiment, the imaging unit 850 may be an RGB camera configured to acquire images or a video stream at a high framerate, e.g., 120 frames per second. The camera may be operatively connected to the controller 820. Thus, the coherent light system may alternately capture a coherent light image and an incoherent light image, e.g., a white light image. The video processing module 830 may, for example, output a video stream based on the incoherent light images with a framerate of 60 fps at the video signal output 834, and may send a video stream based on the coherent light images to display 840, also with a framerate of 60 fps. In some embodiments, images acquired when the optical switch was switching between a first state and a second state may be of lower quality. In that case, the camera may e.g., acquire a video stream comprising sequences of an incoherent light image, a switching image, a coherent light image, and another switching image. The video processing module may disregard the switching images, and generate video streams based on coherent light and on incoherent light at 30 fps each.

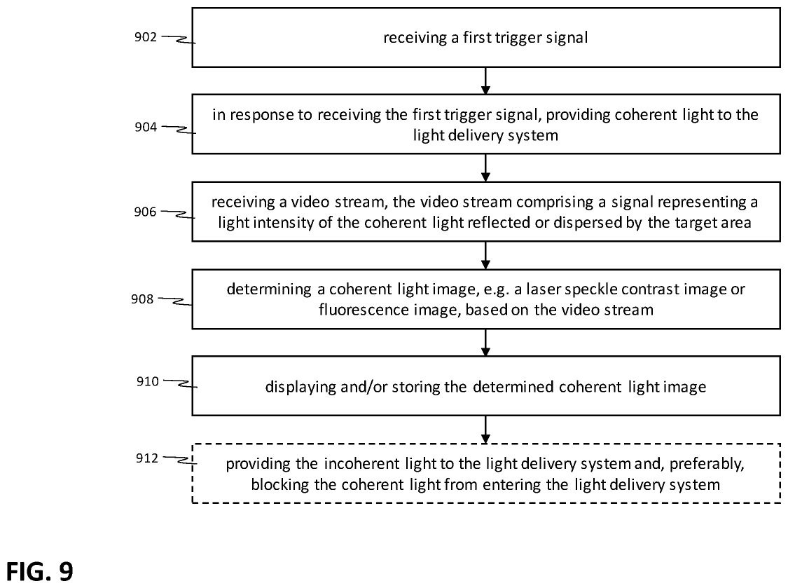

FIG. 9 depicts a method for generating a coherent light image using a coherent light coupling system according to an embodiment of the invention. The coherent light coupling system may comprise an optical input for receiving incoherent light from a light source of an endoscopic system, a first coherent light source for generating coherent light of a first wavelength, and an optical output for alternately providing the incoherent light and the coherent light of the first wavelength to a light delivery system of the endoscopic system. The optical output may be connect to the endoscope via a first light guide and the optical input may be connected to the incoherent light source via a second light guide.

The coherent light coupling system may further comprise an optical switch configured for switching between a first state wherein the incoherent light is injected into the optical output and the coherent light is not injected into the optical output and, hence, not into the first light guide, and a second state wherein the coherent light is injected into the optical output and the incoherent light is not injected into the optical output and, hence, into the first light guide.

In a first step 902, a controller in the coherent light coupling system may receive a first trigger signal. In response to receiving the first trigger signal, the controller may cause the optical switch to switch 904 to the second state. Thus, coherent light may be provided to the light delivery system of the endoscopic system. Consequently, the endoscope may illuminate a target area with coherent light, and collect light reflected or emitted by tissue in the target area in response to being illuminated. A camera of the endoscopic system may capture a video stream based on the collected light.

In a next step 906, a video processing module in the coherent light coupling system may receive the video stream from the camera of the endoscopic system, the video stream comprising image information based on illuminating the target area with coherent light of the first wavelength. Subsequently, the video processing module may determine 908 one or more coherent light images, e.g., laser speckle contrast images or fluorescence images such as ICG-based images, based on the received video stream. In an embodiment, the video stream may comprise frames or the video processing module may be configured to determine frames based on the received video stream. A single coherent light image may be based on a plurality of frames of the received video stream.

In a next step 910, the coherent light coupling system may provide the one or more determined coherent light images to a video signal output of the coherent light coupling system, display the one or more determined coherent light images on a display of the coherent light coupling system and/or store the one or more determined coherent light images in a database included in or connected to the coherent light coupling system.

In an optional step 912, the coherent light coupling system may cause the optical switch to switch to the first state after a predetermined number of coherent light images has been determined, after a predetermined amount of time, or upon receiving a second trigger signal.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

1. A coupling device for coupling coherent light and incoherent light into a first light guide of a light delivery system of an endoscopic system, the endoscopic system comprising an incoherent light source for generating the incoherent light, the coupling device comprising:
an optical input comprising an optical input connector configured to releasably connect the coupling device to the incoherent light source via a second light guide, the optical input being configured for receiving the incoherent light from the endoscopic system;
a first light source configured to generate coherent light of a first wavelength, or a coherent light input configured to receive coherent light of the first wavelength;
an optical output comprising an optical output connector configured to releasably connect the coupling device to an endoscope via the first light guide, the optical output being configured for providing the incoherent light and the coherent light of the first wavelength to the first light guide;
in at least a first state of the coupling device, the coupling device is configured to be an unobstructed incoherent light beam path for propagating the incoherent light from the optical input to the optical output;
in at least a second state of the coupling device, the coupling device is configured to be an unobstructed coherent light beam path for propagating the coherent light from the coherent light source or from the coherent light input to the optical output; and
an optical coupler configured to, simultaneously and/or alternately, inject the incoherent light and the coherent light of the first wavelength into the first light guide, wherein the coherent light and the incoherent light have a shared optical path at the optical output.

2. The coupling device as claimed in claim 1, wherein the optical coupler is an optical switch configured for switching between a first state wherein the coherent light is not injected into the first light guide, and a second state wherein the coherent light is injected into the first light guide.

3. The coupling device as claimed in claim 2, wherein in the first state, the optical switch is configured to inject the incoherent light into the first light guide, and wherein in the second state, the optical switch is configured not to inject the incoherent light into the first light guide.

4. The coupling device as claimed in claim 2, wherein the optical switch comprises:
a first mirror movable between a first position in which a reflective surface of the first mirror is positioned to reflect incoherent light propagating over the incoherent light beam path, and a second position, in which the reflective surface of the first mirror is positioned to reflect the incoherent light in a direction away from the incoherent light beam path; and/or,
a second mirror movable between a first position in which a reflective surface of the second mirror is positioned to reflect coherent light propagating over the coherent light beam path, and a second position, in which the reflective surface of the first mirror is positioned to reflect the coherent light in a direction away from the coherent light beam path.

5. The coupling device as claimed in claim 2, wherein the optical switch comprises:
a first switchable mirror, switchable between a transparent state and a reflective state, positioned to reflect, in the reflective state, incoherent light propagating over the incoherent light beam path; and/or
a second switchable mirror, switchable between a transparent state and a reflective state, positioned to reflect, in the reflective state, coherent light propagating over the coherent light beam path.

6. The coupling device as claimed in claim 2, wherein the optical switch comprises:
a first light beam blocker configured to block the coherent light in the first state.

7. The coupling device as claimed in claim 2, wherein the optical coupler comprises:
a first convergent lens configured to reduce divergence of the incoherent light from the optical input and a second convergent lens configured to focus the incoherent light into the first light guide; and
a mirror, positioned between the first lens and the second lens, configured to steer the coherent light beam and/or the incoherent light beam into the first light guide.

8. The coupling device as claimed in claim 2, wherein the optical coupler comprises:
a tapered light pipe having a wide end for receiving the incoherent light from the optical input and a narrow end for providing the incoherent light to the optical output, the tapered light pipe being divided into a first part and a second part by a plane intersecting the tapered light pipe between the wide end and the narrow end, a normal of the plane defining a non-zero angle with a longitudinal axis of the tapered light pipe; and
a mirror, positioned on the plane between the first part and the second part of the tapered light pipe on the longitudinal axis of the tapered light pipe, configured to steer the laser beam into the first light guide.

9. The coupling device as claimed in claim 2, wherein the optical switch comprises:
an internal light guide configured to guide the incoherent light from a first end of the internal light guide to a second end of the internal light guide; and
a switching body comprising at least the second end of the internal light guide, the switching body being movable between a first position corresponding to the first state and a second position corresponding to the second state,
in which first position, the switching body is configured to block the coherent light beam path, to position a first end of the internal light guide for receiving incoherent light from the optical input, and to position the second end for injecting the incoherent light into the first light guide; and
in which second position, the switching body is configured not to block the coherent light beam path, and to position at least the second end of the internal light guide such that the incoherent light is not injected into the first light guide.

10. The coupling device as claimed in claim 9, wherein the internal light guide is a flexible light guide and wherein the first end of the internal light guide is connected to the optical input.

11. (canceled)

12. The coupling device as claimed in claim 9, wherein the optical switch is an electrically operated switch with a motor, the motor being configured to move the switching body between the first position and the second position via a cam or a crank.

13. (canceled)

14. The coupling device as claimed in claim 2, wherein the optical switch is configured to switch automatically back and forth between the first state and the second state.

15. (canceled)

16. The coupling device as claimed in claim 1, wherein the second light guide is a flexible light guide.

17. The coupling device as claimed in claim 2, wherein the first light source is a narrow-bandwidth laser.

18. (canceled)

19. The coupling device as claimed in claim 2, wherein the first wavelength is in the red part of the electromagnetic spectrum.

20. (canceled)

21. (canceled)

22. The coupling device as claimed in claim 2, wherein the endoscopic system comprises a camera, the camera being configured to provide a video signal, and wherein the coupling device further comprises:
a video signal input connector configured to receive the video signal;
a video signal output connector configured to provide a video output; and
an image processing module configured to generate coherent light images.

23. (canceled)

24. (canceled)

25. (canceled)

26. (canceled)

27. (canceled)

28. (canceled)

29. The coupling device of claim 2 and further comprising an image processing system configured to generate coherent light images with the endoscopic system, the endoscopic system comprising a video processing unit, and an endoscope, the endoscope being connected to a camera, the image processing system comprising video processing device, the video processing device comprising:
a video signal input connector configured to receive a video signal from the camera;
a first video signal output connector configured to provide a video output, the first video signal output connector configured to be connected to a video signal input of the video processing unit of the endoscopic system; and
an image processing module for generating coherent light images based the video signal when the optical switch is in the first state.

30. The coupling device as claimed in claim 29, wherein the image processing system further comprises a second video signal output connector configured to be connected to a second display, the image processing system being configured to provide an incoherent light image to the first video output when the optical switch is in the first state and to provide a coherent light image to the second video signal output when the optical switch is in the second state.

31. (canceled)

32. (canceled)

33. (canceled)

34. A method for generating a coherent-light-based image of a target area in an object using an endoscopic system, the endoscopic system comprising:
an incoherent light source for generating incoherent light; and,
an endoscope, the endoscope comprising an insertion tube for inserting in the patient body, a light delivery system for illuminating the target area, and an image sensor for acquiring an image of the target area;
the endoscope being releasably connected to a coherent light coupling system via a first light guide, the coherent light coupling system being configured to:
generate or receive a trigger signal;
receive the incoherent light from the incoherent light source via a second light guide;
receive or generate coherent light of a first wavelength; and
selectively provide the coherent light and/or the incoherent light to the first light guide, wherein the coherent light and the incoherent light have a shared optical path in the first light guide;
the method comprising:
receiving a first trigger signal;
in response to receiving the first trigger signal, providing coherent light to the light delivery system;
receiving a video stream, the video stream comprising a signal representing a light intensity of the coherent light reflected or dispersed by the target area; and
determining a coherent light image based on the video stream.

35. (canceled)

36. (canceled)

37. (canceled)

38. (canceled)

39. The coupling device as claimed in claim 6 wherein the optical switch comprises a second light beam blocker configured to block the incoherent light in the second state.
